(19) United States
(12) Patent Application Publication
(10) Pub. No.: US 2009/0048283 A1
(43) Pub. Date: Feb. 19, 2009

(54) Salt of an androgen receptor modulator

(76) Inventor: Claire H. Lee, Yonkers, NY (US)

Correspondence Address: MERCK AND CO., INC, P O BOX 2000, RAHWAY, NJ, 07065-0907, US

(21) Appl. No.: 12/228,356

(22) Filed: Aug. 12, 2008

Related U.S. Application Data
(60) Provisional application No. 60/964,824, filed on Aug. 15, 2007.

(51) Int. Cl. A61K 31/435; C07D 221/18; A61P 25/00

(52) U.S. Cl. 514/284; 546/77

(57) ABSTRACT

The biphthalate salts of compound I are modulators of the androgen receptor (AR) in a tissue selective manner. These compounds are useful in the enhancement of weakened muscle tone and the treatment of conditions caused by androgen deficiency or which can be ameliorated by androgen administration, including osteoporosis, osteopenia, glucocorticoid-induced osteoporosis, periodontal disease, bone fracture, bone damage following bone reconstructive surgery, sarcopenia, frailty, aging skin, male hypogonadism, postmenopausal symptoms in women, atherosclerosis, hypercholesterolemia, hyperlipidemia, obesity, aplastic anemia and other hematopoietic disorders, inflammatory arthritis and joint repair, HIV-wasting, prostate cancer, benign prostatic hyperplasia (BPH), abdominal adiposity, metabolic syndrome, type II diabetes, cancer cachexia, Alzheimer's disease, muscular dystrophies, cognitive decline, sexual dysfunction, sleep apnea, depression, premature ovarian failure, and autoimmune disease, alone or in combination with other active agents.

### CROSS-REFERENCE TO RELATED APPLICATIONS

This application claims priority from U.S. Provisional Application Ser. No. 60/964,824, filed Aug. 15, 2007.

### FIELD OF THE INVENTION

The present invention relates to crystalline forms including, polymorphs and psuedopolymorphs of the biphthalate salt of N-(3H-imidazo[4,5-B]pyridin-2-ylmethyl)-2-fluoro-4-methyl-3-oxo-4-aza-5-alpha-androst-1-en-17-beta-carboxamide, a tissue-selective androgen receptor modulator (SARM). This SARM is thereby useful for the treatment of conditions caused by androgen deficiency or which can be ameliorated by androgen administration, such as osteoporosis, periodontal disease, bone fracture, frailty, and sarcopenia. Additionally, the SARM of the present invention can be used to treat mental disorders associated with low testosterone, such as depression, sexual dysfunction, and cognitive decline. The SARM, being an antagonist in specific tissues, may also be useful in conditions where elevated androgen tone or activity causes symptoms, such as benign prostate hyperplasia and sleep apnea.

### BACKGROUND OF THE INVENTION

The androgen receptor (AR) belongs to the superfamily of steroid/thyroid hormone nuclear receptors, whose other members include the estrogen receptor, the progesterone receptor, the glucocorticoid receptor, and the mineralocorticoid receptor. The AR is expressed in numerous tissues of the body and is the receptor through which the physiological as well as the pathophysiological effects of androgens, such as testosterone (T) and dihydrotestosterone (DHT), are mediated. Structurally, the AR is composed of three functional domains: the ligand binding domain (LBD), the DNA-binding domain, and amino-terminal domain. A compound that binds to the AR and mimics the effects of an endogenous AR ligand is referred to as an AR agonist, whereas a compound that inhibits the effects of an endogenous AR ligand is termed an AR antagonist.

Androgen ligand binding to the AR induces a ligand/receptor complex, which, after translocation into the nucleus of the cell, binds to regulatory DNA sequences (referred to as androgen response elements) within the promoter or enhancer regions of the target genes present in the nucleus. Other proteins termed cofactors are next recruited, which bind to the receptor leading to gene transcription.

Androgen therapy has been used to treat a variety of male disorders such as reproductive disorders and primary or secondary male hypogonadism. Moreover, a number of natural or synthetic AR agonists have been investigated for the treatment of musculoskeletal disorders, such as bone disease, hematopoietic disorders, neuromuscular disease, rheumatological disease, wasting disease, and for hormone replacement therapy (HRT), such as female androgen deficiency. In addition, AR antagonists, such as flutamide and bicalutamide, are used to treat prostate cancer. It would therefore be useful to have available compounds that can activate (“agonize”) the function of the AR in a tissue-selective manner that would produce the desired osteo- and myoanabolic effects of androgens without the negative androgenic properties, such as virilization and repression of high density lipoprotein cholesterol (HDL).

The beneficial effects of androgens on bone in postmenopausal osteoporosis were documented in recent studies using combined testosterone and estrogen administration [Hofbauer, et al., Eur. J. Edocrinol. 140: 271-286 (1999)]. In a large 2-year, double-blind comparison study, oral conjugated estrogen (CEE) and methyltestosterone combinations were demonstrated to be effective in promoting accrual of bone mass in the spine and hip, while conjugated estrogen therapy alone prevented bone loss [J. Reprod. Med., 44: 1012-1020 (1999)].

Additionally, there is evidence that hot flushes decrease in women treated with CEE and methyltestosterone; however, 30% of the treated women suffered from significant increases in acne and facial hair, a complication of all current androgen pharmacotherapies [Watts, et al., Obstet. Gynecol., 85: 529-537 (1995)]. It was also found that the addition of methyltestosterone to CEE decreased HDL levels, as seen in other studies. Thus, the virilizing potential and effects on lipid profile of current androgen therapies provide a rationale for developing tissue-selective androgen receptor agonists.

Androgens play an important role in bone metabolism in men [Anderson, et al., “Androgen supplementation in eugonadal men with osteoporosis—effects of six months of treatment on bone mineral density and cardiovascular risk factors,” Bone, 18: 171-177 (1996)]. Even in eugonadal men with osteoporosis, the therapeutic response to testosterone treatment reveals that androgens exert important osteoanabolic effects. Mean lumbar BMD increased from 0.799 gm/cm² to 0.839 g/cm², in 5 to 6 months in response to 250 mg of testosterone ester administered intramuscularly. SARMs can thus be used to treat osteoporosis in men.

Androgen deficiency occurs in men with stage D prostate cancer (metastatic) who undergo androgen deprivation therapy (ADT). Endocrine orchiectomy is achieved by long acting GnRH agonists, while androgen receptor blockade is implemented with AR antagonists. In response to hormonal deprivation, these men suffered from hot flushes, significant bone loss, weakness, and fatigue. In a pilot study of men with stage D prostate cancer, osteopenia (50% vs. 38%) and osteoporosis (38% vs. 25%) were more common in men who had undergone ADT for greater than one year than the patients who did not undergo ADT [Wei, et al., Urology, 54: 607-611 (1999)]. Lumbar spine BMD was significantly lower in men who had undergone ADT. Thus tissue selective AR antagonists in the prostate that lack antagonistic action in bone and muscle can be useful agents for the treatment of prostate cancer, either alone or as an adjunct to traditional ADT [See also A. Stoch, et al., J. Clin. Endocrin. Metab., 86: 2787-2791 (2001)].

Tissue-selective AR antagonists can also treat polycystic ovarian syndrome in postmenopausal women. See C. A. Eagleson, et al., “Polycystic ovarian syndrome: evidence that flutamide restores sensitivity of the gonadotropin-releasing hormone pulse generator to inhibition by estradiol and progesterone,” J. Clin. Endocrinol. Metab., 85: 4047-4052 (2000).

SARMs can also treat certain hematopoietic disorders as androgens stimulate renal hypertrophy and erythropoietin (EPO) production. Prior to the introduction of recombinant human EPO, androgens were employed to treat anemia caused by chronic renal failure. In addition, androgens increase serum EPO levels in anemic patients with non-severe aplastic anemia and myelodysplastic syndromes. Treatment for anemia will require selective action such as can be provided by SARMs.

SARMs can also have clinical value as an adjunct to the treatment of obesity. This approach to lowering body fat is supported by published observations that androgen administration reduced subcutaneous and visceral fat in obese patients [J. C. Lovejoy, et al., “Oral anabolic steroid treatment, but not parenteral androgen treatment, decreases abdominal fat in obese, older men,” Int. J. Obesity, 19: 614-624 (1995)], [J. C. Lovejoy, et al., “Exogenous Androgens Influence Body Composition and Regional Body Fat Distribution in Obese Postmenopausal Women—A Clinical Research Center Study,” J. Clin. Endocrinol. Metab., 81: 2198-2203 (1996)]. Therefore, SARMs devoid of unwanted androgenic effects can be beneficial in the treatment of obesity.

Androgen receptor agonists can also have therapeutic value against metabolic syndrome (insulin resistance syndrome, syndrome X), particularly in men. Low levels of total and free testosterone and sex hormone-binding globulin (SHBG) in men have been associated with type 2 diabetes, visceral obesity, insulin resistance (hyperinsulinemia, dyslipidemia) and metabolic syndrome. [D. Laaksonen, et al., Diabetes Care, 27 (5): 1036-1041(2004); see also D. Laaksonen, et al. Euro. J Endocrin, 149: 601-608 (2003); P. Márin, et al. Int. J. Obesity, 16: 991-997 (1992), and P. Márin, et al. Obesity Res., 1(4): 245-251 (1993)].

Androgen receptor agonists can also have therapeutic value against neurodegenerative diseases such as Alzheimer's disease (AD). The ability of androgens to induce neuroprotection through the androgen receptor was reported by J. Hammond, et al., “Testosterone-mediated neuroprotection through the androgen receptor in human primary neurons,” J. Neurochem., 77: 1319-1326 (2001). Gouras et al. reported that testosterone reduces secretion of Alzheimer's β-amyloid peptides and can therefore be used in the treatment of AD [(Proc. Nat. Acad. Sci., 97: 1202-1205 (2000)]. A mechanism via inhibition of hyperphosphorylation of proteins implicated in the progression AD has also been described [S. Papasozomenos, “Testosterone prevents the heat shock-induced over activation of glycogen synthase kinase-3β but not of cyclin-dependent kinase 5 and c-Jun NH2-terminal kinase and concomitantly abolishes hyperphosphorylation of τ: Implications for Alzheimer's disease,” Proc. Nat. Acad. Sci., 99: 1140-1145 (2002)].

Androgen receptor agonists can also have a beneficial effect on muscle tone and strength. Recent studies have demonstrated that “physiologic androgen replacement in healthy, hypogonadal men is associated with significant gains in fat-free mass, muscle size and maximal voluntary strength” [S. Bhasin, et al., J. Endocrin., 170: 27-38 (2001)].

Androgen receptor modulators can be useful in treating decreased libido in both men and women. Androgen deficiency in men is related to diminished libido. [S. Howell et al., Br. J. Cancer, 82: 158-161]. Low androgen levels contribute to the decline in sexual interest in many women during their later reproductive years. [S. Davis, J. Clin. Endocrinol. Metab., 84: 1886-1891 (1999)]. In one study, circulating free testosterone was positively correlated with sexual desire. Id. In another study, women with primary or secondary adrenal insufficiency were provided physiological DHEA replacement (50 mg/day). Compared with women taking placebo, DHEA-administered women showed an increase in the frequency of sexual thoughts, interest, and satisfaction. [W. Arlt, et al., N Engl. J. Med. 341:1013-1020 (1999), see also, K. Miller, J. Clin. Endocrinol. Metab., 86: 2395-2401 (2001)].

Additionally, androgen receptor modulators may also be useful in treating cognitive impairment. In a recent study, high-dose oral estrogen either alone or in combination with high-dose oral methyltestosterone was given to postmenopausal women for a four-month period. Cognitive tests were administered before and after the four-month hormone treatment. The investigation found that women receiving a combination of estrogen (1.25 mg) and methyltestosterone (2.50 mg) maintained a steady level of performance on the Building Memory task, but the women receiving estrogen (1.25 mg) alone exhibited decreased performance. [A. Wisniewski, Horm. Res. 58:150-155 (2002)].

Compound I, N-(3H-imidazo[4,5-B]pyridin-2-ylmethyl)-2-fluoro-4-methyl-3-oxo-4-aza-5-alpha-androst-1-en-17-beta-carboxamide, has the following structural formula:

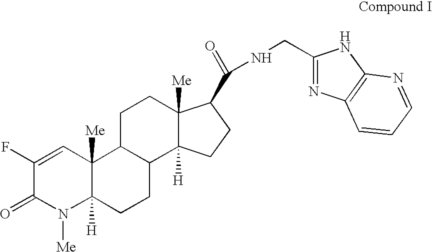

This compound as well as various solid state forms have been previously disclosed in U.S. Pat. No. 7,186,838 and PCT international application PCT US2006/046263, respectively.

### SUMMARY OF THE INVENTION

The present invention relates to crystalline, polymorphic, and pseudopolymorphic solid state forms of the biphthalate salt of compound I, N-(3H-imidazo[4,5-B]pyridin-2-ylmethyl)-2-fluoro-4-methyl-3-oxo-4-aza-5-alpha-androst-1-en-17-beta-carboxamide, its use, and pharmaceutical compositions. The present invention additionally provides processes for the preparation of crystalline solid state forms and hydrates of the biphthalate salt of compound I, N-(3H-imidazo[4,5-B]pyridin-2-ylmethyl)-2 fluoro-4-methyl-3-oxo-4-aza-5-alpha-androst-1-en-17-beta-carboxamide.

Knowledge of the various physicochemical properties of an active material is important to optimize the utility of that material in all aspects of a compound's life cycle including, for example, its manufacture and pharmaceutical processing, and/or its storage, shipping, and therapeutic uses. Sometimes a pharmacologically active compound can not be fashioned into a suitable pharmaceutical composition because the active compound has unfavorable physical properties such as, for example, poor milling properties or poor dissolution properties.

One aspect of the present invention provides crystalline solid state forms and hydrates of the biphthalate salt of compound I, N-(3H-imidazo[4,5-B]pyridin-2-ylmethyl)-2-fluoro-4-methyl-3-oxo-4-aza-5-alpha-androst-1-en-17-beta-carboxamide. The crystalline solid forms and hydrates of the present invention have good physiochemical properties; desirable medicinal properties; and good processing properties. Certain embodiments of the present invention exhibit desirable stability characteristics. The crystalline solid state and hydrate forms of the present invention can be incorporated into a variety of different formulation vehicles making them particularly suitable for pharmaceutical utility.

The solid state forms of compound I are effective as androgen receptor agonists and are particularly effective as SARMs. The polymorphic, psuedopolymorphic, and amorphous solid state forms of the present invention are, therefore useful for the treatment of conditions caused by androgen deficiency or which can be ameliorated by androgen administration.

### BRIEF DESCRIPTION OF THE FIGURES

FIG. 1 is a characteristic X-ray pattern of Form B, the crystalline dihydrate 2:1 compound I:biphthalate salt.

FIG. 2 is a typical DSC curve of Form B, the crystalline dihydrate 2:1 compound I:biphthalate salt.

FIG. 3 is a solid-state carbon-13 nuclear magnetic resonance (NMR) spectrum of Form B, the crystalline dihydrate 2:1 compound I:biphthalate.

FIG. 4 is a typical thermogravimetric (TG) curve of Form B, the crystalline dihydrate 2:1 compound I:biphthalate salt.

FIG. 5 is an overlay of variable temperature X-ray powder diffraction patterns showing the anhydrous crystalline 2:1 compound I:biphthalate salt, Form C, which was not isolatable.

FIG. 6 is an overlay of X-ray powder diffraction patterns showing Form D, the tetrahydrate 2:1 compound I:biphthalate salt.

### DETAILED DESCRIPTION OF THE INVENTION

The present invention provides polymorphic and pseudopolymorphic forms including crystalline, solvates, hydrates, and anhydrates, of the 2:1 compound I:biphthalate salt of N-(3H-imidazo[4,5-B]pyridin-2-ylmethyl)-2-fluoro-4-methyl-3-oxo-4-aza-5-alpha-androst-1-en-17-beta-carboxamide:

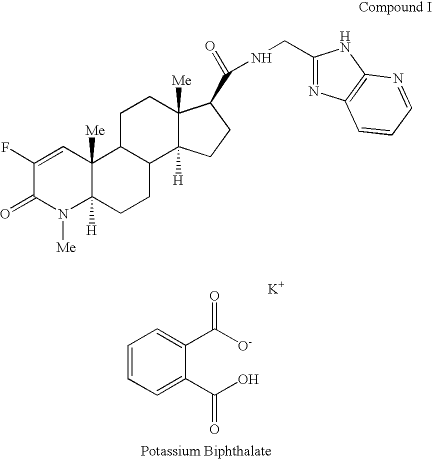

The present invention also relates to pharmaceutical compositions comprising solid-state forms of the compound of the present invention and a pharmaceutically acceptable carrier.

Polymorphs are two or more crystalline phases of the same chemical compound that possess a different arrangement and/or conformation of the molecules in a crystal lattice. Different polymorphs of an active pharmaceutical compound can exhibit different physical and chemical properties. Different physical and chemical properties of a given active pharmaceutical compound can also depend on moisture/solvent content. Pseudopolymorphs include hydrates and solvates of a compound. Pseudopolymorphs incorporate one or more solvents or water into the crystal lattice of the compound.

The solid state physical properties of a compound can be influenced by controlling the conditions under which the compounds are obtained in solid form. Different crystal structures of a compound may exhibit variations in physical and chemical properties such as color, stability, processability, dissolution and even bioavailability. For example, flowability, which affects how particles flow past each other, is one such solid state physical property that can affect how a compound is milled. To compensate for poor flowability and obtain a millable pharmaceutical tablet, often times a pharmaceutical formulator must add other agents to a pharmaceutical composition such as talc or starch, to act as glidants or lubricants. It would be beneficial to minimize use of such agents by choosing a solid form of an active ingredient that exhibits favorable flowability and milling properties.

Another solid state physical property of a compound is the rate of dissolution in a fluid, such as water. The dissolution of an active ingredient may affect the storage stability of a pharmaceutical product. Additionally, the dissolution rate of an active ingredient affects the rate at which an orally-administered active ingredient can reach a subject's bloodstream. Choice of a suitable solid form can maximize the storage stability and/or the dissolution rate of an active ingredient.

Additionally, identification, manufacture, and control of specific solid state forms are of interest to the pharmaceutical industry during clinical trials or other research studies. If a given polymorphic form of a drug is not held constant during clinical studies, the exact dosage form being studied may not be comparable from one lot to another introducing an uncontrolled source of error to the study.

Generally, it is difficult to predict whether or not a given compound will form various crystalline solid state forms. It is even more difficult to predict the structural configurations and the physical properties of these different crystalline solid state forms.

It would be advantageous to identify and utilize the various polymorphs, pseudopolymorphs, and amorphous forms of a pharmaceutically active compound in order to create an arsenal of solid forms with varying physical properties. In this manner, a pharmaceutical dosage form can be designed with a specific desirable property, such as for example, a specific dissolution rate, milling property, thermal stability or shelf-life.

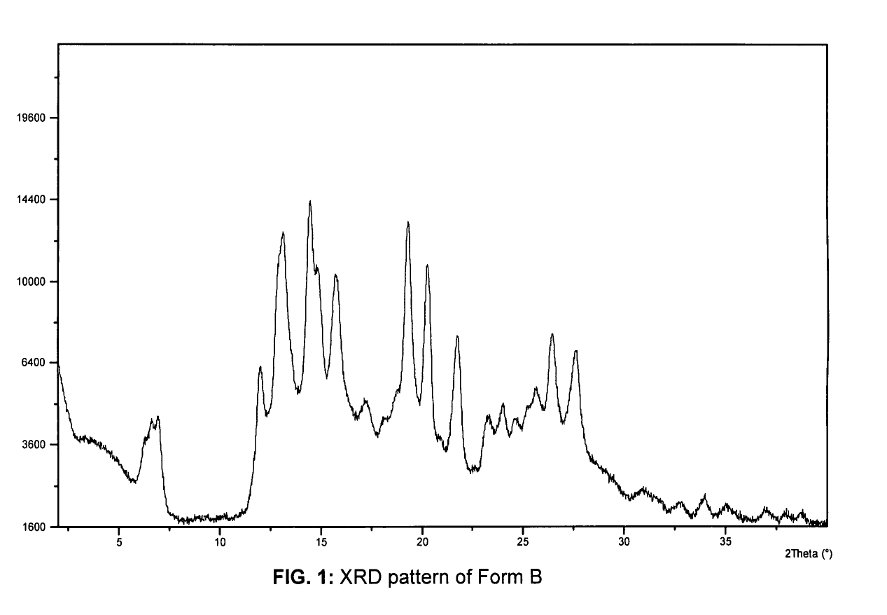

FIG. 1 is a characteristic X-ray pattern of Form B, a crystalline dihydrate 2:1 compound I:biphthalate salt of N-(3H-imidazo[4,5-B]pyridin-2-ylmethyl)-2-fluoro-4-methyl-3-oxo-4-aza-5-alpha-androst-1-en-17-beta-carboxamide having a X-ray powder diffraction (XRPD) pattern with characteristic reflections corresponding to d-spacings of 5.65±0.1, 4.62±0.1, and 4.40±0.1 angstroms. The crystalline dihydrate, Form B, is further characterized by the additional reflections corresponding to d-spacings of 7.41±0.1, 4.10±0.1, and 3.38±0.1 angstroms. The dihydrate crystalline Form B is still further characterized by reflections corresponding to d-spacings of 3.84±0.1, 3.72±0.1, and 3.24±0.1 angstroms.

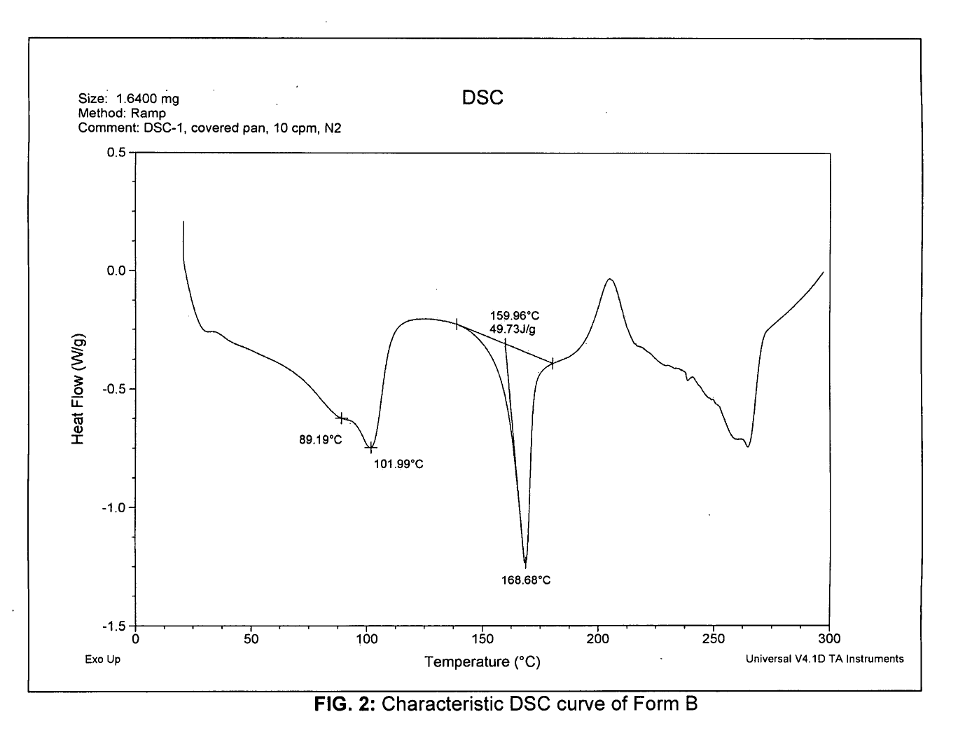

FIG. 2 is a typical differential scanning calorimetry (DSC) curve of Form B, the crystalline dihydrate 2:1 compound I:biphthalate salt. The crystalline dihydrate 2:1 compound I:biphthalate salt exhibited endotherms below 110° C. (peaks at 89.2±5° C. and 102.0±5° C.) or the endotherm may appear as one broad peak ranging from about 85±5° C. to about 105±5° C. The DSC endotherms correspond to the weight loss observed in thermogravimetiric analysis (TGA). The crystalline dihydrate, Form B, is further characterized by an endotherm due to melting, exhibiting an onset temperature of about 160.0° C., and a peak temperature of about 162±10° C., such as, for example, 168.7±0.5° C. The crystalline dihydrate, Form B, is further characterized by an enthalpy change of about 49.7 Joules per gram (J/gm).

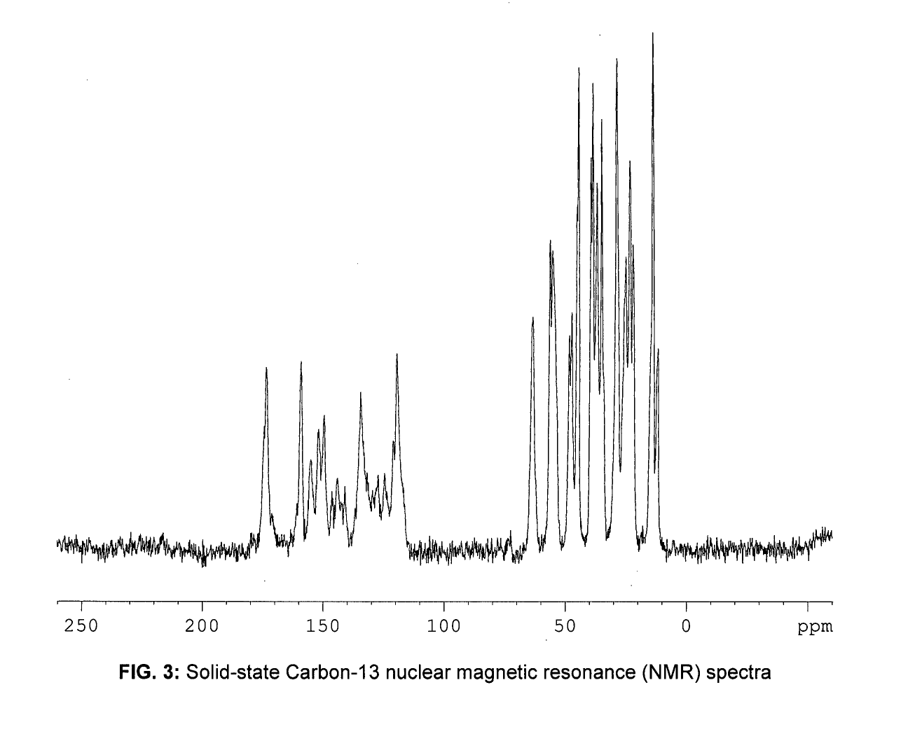

FIG. 3 is a solid-state Carbon-13 nuclear magnetic resonance (NMR) spectrum of the crystalline dihydrate 2:1 compound I:biphthalate salt, Form B. The crystalline dihydrate 2:1 compound I:biphthalate salt exhibited characteristic signals with chemical shift values of 13.8±0.1, 44.4±0.1, and 119.4±0.1 p.p.m. Form B is further characterized by signals with chemical shift values of 28.7±0.1, 56.2±0.1, and 173.3±0.1 p.p.m. The crystalline dihydrate 2:1 compound I:biphthalate salt is even further characterized by signals with chemical shift values of 38.6±0.1, 22.1±0.1, and 158.9±0.1 p.p.m.

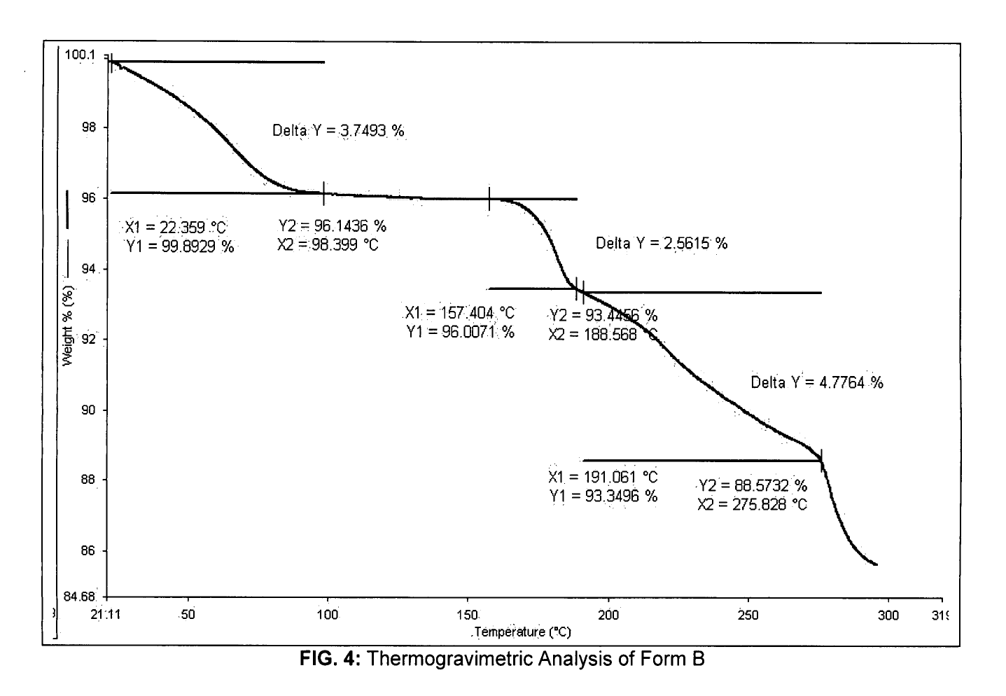

FIG. 4 reflects a typical thermogravimetric analysis (TGA) curve for the crystalline dihydrate 2:1 compound I:biphthalate salt. As shown in FIG. 4, the TGA exhibits a one-step weight loss based on initial weight of the sample (about 3.7% to about 98° C.) prior to melting at about 169° C.

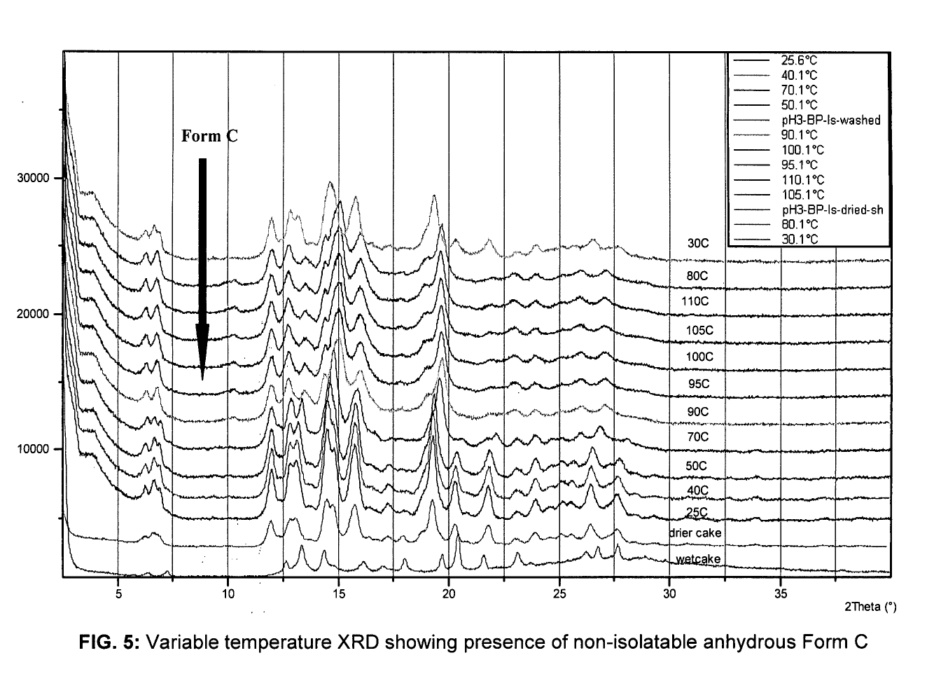

FIG. 5 is an overlay of variable temperature X-ray powder diffraction patterns showing the appearance of the non-isolatable anhydrous Form C of the 2:1 compound I:biphthalate salt. The transition to dehydrated state was found to be form about 95 to about 100° C. The variable temperature x-ray stage was not equipped with N₂ flow and cooling to 30° C. was accompanied by adsorption and X-ray pattern changed back to Type B, since Form C readily hydrates to Form B in ambient conditions.

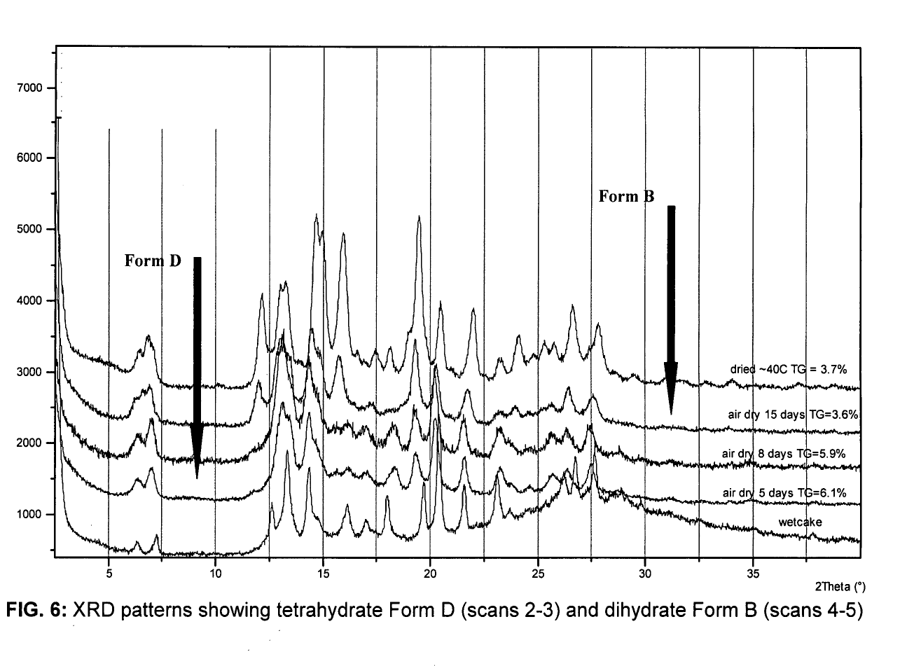

FIG. 6 is an overlay of X-ray powder diffraction patterns comparing Form B, the dihydrate 2:1 compound I:biphthalate salt, with Form D, the tetrahydrate 2:1 compound I:biphthalate salt. When the wet cake of the 2:1 compound I:biphthalate salt, was air dried at room temperature, Form D was obtained at both T=5 days and T=8-days. The various methods of drying possible, in addition to the size of the sample to be dried, can vary the time needed for drying. The dryness was determined by a stable TG curve showing about a 6% weight loss. Here, the method was to dry in open air, and at both time points (T=5 and T=8 days), the weight loss by TGA was equal to about 6%. Form D converts to Form B upon further drying.

One embodiment of the invention relates to a process for making the crystalline Form B of the biphthalate salt of compound I comprising: a) dissolving the compound, N-(3H-imidazo[4,5-B]pyridin-2-ylmethyl)-2-fluoro-4-methyl-3-oxo-4-aza-5-alpha-androst-1-en-17-beta-carboxamide, in a potassium biphthalate and sulfuric acid mixture having a pH ranging from about 2 to about 9, to form a mixture; b) agitating the mixture until crystallization is complete and solids are formed; c) isolating the solids; and d) drying the solids.

In one embodiment of the invention, isolating the solids further comprises filtering of the solids via a vacuum filter and washing the solids with at least one wash with a solvent consisting of an aqueous solution including, but not limited to, water, 1,2-dimethoxyethane, isopropyl alcohol, isopropyl acetate, and acetone.

In another embodiment of the invention, the solvent is water.

In one embodiment of the invention, the process for making the crystalline Form B of the biphthalate salt of compound I comprises: a) dissolving the compound, N-(3H-imidazo[4,5-B]pyridin-2-ylmethyl)-2-fluoro-4-methyl-3-oxo-4-aza-5-alpha-androst-1-en-17-beta-carboxamide, in a biphthalate solution (ammonium or potassium biphthalate) having a pH ranging from about 2 to about 9, to form a mixture; b) agitating the mixture until crystallization is complete and solids are formed; c) isolating the solids; and d) drying the solids.

In one embodiment of the invention, an anhydrous 2:1 compound I:biphthalate salt, Form C, was observed with variable temperature X-ray. (FIG. 4). The transition to the dehydrated state was found to be between around 95 to 100° C. The variable temperature X-ray stage was not equipped with N₂ flow; therefore, cooling to around 30° C. was accompanied by adsorption and a return to Form B, the most stable form.

In another embodiment of the invention, anhydrous 2:1 compound I:biphthalate material was non-isolatable at ambient condition, but the hygroscopicity study indicated the presence of one or more hydrated states from 15% to 95% RH. The weight percent of water was 3.4% at 15% RH with a gradual increase to 6.1% at 95%. When the wet cake of the 2:1 compound I:biphthalate salt of N-(3H-imidazo[4,5-B]pyridin-2-ylmethyl)-2-fluoro-4-methyl-3-oxo-4-aza-5-alpha-androst-1-en-17-beta-carboxamide was dried at room temperature, a distinct XRD pattern appeared compared to that of the dihydrate Form B. At both 5 days and 8 days the additional form, Form D, had a weight loss by TG of 6%. Form D is suspected to be a tetrahydrate biphthalate salt based on its TG data in addition to the hygroscopicity data of Form B. Upon further drying (15 days), the TG and XRD data conform to that of Form B, the dihydrate 2:1 compound I:biphthalate salt.

X-ray powder diffraction studies are widely used to characterize molecular structures, crystallinity, and polymorphism. The X-ray powder diffraction (XRPD) pattern of the crystalline form of the biphthalate salt of the compound I was generated on a Philips Analytical X'Pert PRO X-ray Diffraction System with PW3040/60 console. A PW3373/00 ceramic Cu LEF X-ray tube K-Alpha radiation was used as the source.

In addition to the X-ray powder diffraction patterns described above, the crystalline forms of the biphthalate salts of compound I were further characterized by using a TA Instruments DSC 2910 or equivalent instrumentation for Differential Scanning Calorimetry (DSC). Between 1 and 5 mg sample was weighed into an open pan and a lid was placed lightly over the pan to cover the sample. This covered pan was then placed at the sample position in the calorimeter cell. An empty pan with lid was placed at the reference position. The calorimeter cell was closed and a flow of nitrogen was passed through the cell. The heating program was set to heat the sample at a heating rate of 10° C./min to a temperature of approximately 300° C. The heating program was started. When the run was completed, the data was analyzed using the DSC analysis program contained in the system software. The melting endotherm was integrated between baseline temperature points that are above and below the temperature range over which the endotherm was observed. The data reported are the onset temperature, peak temperature and enthalpy.

In addition to X-ray powder diffraction and differential scanning calorimetry, the crystalline forms of the biphthalate salt of compound I were further characterized by solid-state carbon-13 nuclear magnetic resonance (NMR) spectra. The solid-state carbon-13 NMR spectra were obtained on a Bruker DSX 500WB NMR system using a Bruker 4 mm H/X/Y CPMAS probe. The carbon-13 NMR spectra utilized proton/carbon-13 cross-polarization magic-angle spinning with variable-amplitude cross polarization, total sideband suppression, and SPINAL decoupling at 100 kHz. The samples were spun at 10.0 kHz, and a total of 4098 scans were collected with a recycle delay of 5 seconds. A line broadening of 10 Hz was applied to the spectra before FT was performed. Chemical shifts are reported on the TMS scale using the carbonyl carbon of glycine (176.03 p.p.m.) as a secondary reference.

Thermogravimetric (TG) data was acquired using a Perkin Elmer Model TGA 7 or equivalent instrumentation. Experiments were performed under a flow of nitrogen and using a heating rate of 10° C. min⁻¹ to a maximum temperature of approximately 300° C. After automatically taring the balance, 0.5 to 5 mg of sample was added to the platinum pan, the furnace was raised, and the heating program started. Weight/temperature data were collected automatically by the instrument. Analysis of the results was carried out by selecting the Delta Y function within the instrument software and choosing the temperatures between which the weight loss was to be calculated. Weight losses were reported up to the onset of decomposition/evaporation.

In one embodiment, a polymorph/pseudopolymorph in accordance with the present invention is “substantially pure” and has a polymorphic/pseudomorphic purity of about at least 85%. That is, with reference to a particular crystalline form, the crystalline form comprises less than about 15% by weight of impurities, including other polymorphic, amorphous, and/or pseudopolymorphic forms. In a variant of this embodiment, the polymorphic/psuedopolymorphic purity is at least 95%.

The solid-state forms of the present invention can include a mixture of two or more forms. The mixtures of the solid-state forms in accordance with the present invention will have X-ray diffraction peaks characteristic of each of the various polymorphic forms present in the mixture.

The solid-state forms of the compound of the present invention identified as a SARM are useful to treat diseases or conditions caused by androgen deficiency which can be ameliorated by androgen administration. Such amorphous solvates and crystalline forms are ideal for the treatment of osteoporosis in women and men as a monotherapy or in combination with inhibitors of bone resorption, such as bisphosphonates, estrogens, SERMs, cathepsin K inhibitors, αvβ3 integrin receptor antagonists, calcitonin, and proton pump inhibitors. They can also be used with agents that stimulate bone formation, such as parathyroid hormone or analogs thereof. The various solid forms of the SARM compound of the present invention can also be employed for treatment of prostate disease, such as prostate cancer and benign prostatic hyperplasia (BPH). Moreover, the solid state forms of the compound of this invention exhibit minimal effects on skin (acne and facial hair growth) and can be useful for treatment of hirsutism. Additionally, the biphthalate salt of compound I can stimulate muscle growth and can be useful for treatment of sarcopenia and frailty. They can be employed to reduce visceral fat in the treatment of obesity. Moreover, compounds of this invention can exhibit androgen agonism in the central nervous system and can be useful to treat vasomotor symptoms (hot flushes) and to increase energy and libido. They can also be used in the treatment of Alzheimer's disease.

The solid forms of the biphthalate salt of compound I can also be used in the treatment of prostate cancer, either alone or as an adjunct to GnRH agonist/antagonist therapy, for their ability to restore bone, or as a replacement for antiandrogen therapy because of their ability to antagonize androgen in the prostate, and minimize bone depletion. Further, the compounds of the present invention can be used for their ability to restore bone in the treatment of pancreatic cancer as an adjunct to treatment with antiandrogen, or as monotherapy for their antiandrogenic properties, offering the advantage over traditional antiandrogens of being bone-sparing. Additionally, compounds of this invention can increase the number of blood cells, such as red blood cells and platelets, and can be useful for the treatment of hematopoietic disorders, such as aplastic anemia. Thus, considering their tissue selective androgen receptor agonism listed above, the crystalline forms of the biphthalate salts of compound I are ideal for hormone replacement therapy in hypogonadic (androgen deficient) men.

This invention is also concerned with safely and specifically treating a male subject with abdominal adiposity, metabolic syndrome (also known as the ‘insulin resistance syndrome’ and ‘Syndrome X’), and type II diabetes.

The biphthalate salts of compound I have been found to be tissue-selective modulators of the androgen receptor (SARMs). In one aspect, the solid-state forms of the compound of the present invention can be useful to activate the function of the androgen receptor in a mammal, and in particular to activate the function of the androgen receptor in bone and/or muscle tissue and block or inhibit (“antagonize”) the function of the androgen receptor in the prostate of a male individual or in the uterus of a female individual.

A further aspect of the present invention is the use of the solid-state forms of the biphthalate salts of compound I to attenuate or block the function of the androgen receptor in the prostate of a male individual or in the uterus of a female individual induced by AR agonists, but not in hair-growing skin or vocal cords, and activate the function of the androgen receptor in bone and/or muscle tissue, but not in organs which control blood lipid levels (e.g. liver).

The biphthalate salt of compound I displays submicromolar binding affinity for the androgen receptor and is useful in treating mammals suffering from disorders related to androgen receptor function. Therapeutically effective amounts of the compound, including polymorph(s), pseudopolymorph(s), and amorphous forms, and mixtures thereof, are administered to the mammal, to treat disorders related to androgen receptor function, such as, androgen deficiency, disorders which can be ameliorated by androgen replacement, or which can be improved by androgen replacement, including: enhancement of weakened muscle tone, osteoporosis, osteopenia, glucocorticoid-induced osteoporosis, periodontal disease, bone fracture (for example, vertebral and non-vertebral fractures), bone damage following bone reconstructive surgery, sarcopenia, frailty, aging skin, male hypogonadism, postmenopausal symptoms in women, atherosclerosis, hypercholesterolemia, hyperlipidemia, obesity, aplastic anemia and other hematopoietic disorders, pancreatic cancer, inflammatory arthritis and joint repair, HIV-wasting, prostate cancer, benign prostatic hyperplasia (BPH), cancer cachexia, Alzheimer's disease, muscular dystrophies, cognitive decline, sexual dysfunction, sleep apnea, depression, premature ovarian failure, and autoimmune disease. Treatment is effected by administration of a therapeutically effective amount of the biphthalate salts of compound I to a mammal in need of such treatment. In addition, the solid-state forms of the biphthalate salts of compound I are useful as ingredients in pharmaceutical compositions alone or in combination with other active agents.

In one embodiment, the amorphous and crystalline solid forms of the compound of the present invention can be used to treat conditions in a male individual which are caused by androgen deficiency or which can be ameliorated by androgen replacement, including, but not limited to, osteoporosis, osteopenia, glucocorticoid-induced osteoporosis, periodontal disease, HIV-wasting, prostate cancer, cancer cachexia, obesity, arthritic conditions, anemias, such as for example, aplastic anemia, muscular dystrophies, and Alzheimer's disease, cognitive decline, sexual dysfunction, sleep apnea, depression, benign prostatic hyperplasia (BPH), abdominal obesity, metabolic syndrome, type II diabetes, and atherosclerosis, alone or in combination with other active agents. Treatment is effected by administration of a therapeutically effective amount of the biphthalate salts of compound I to a male individual in need of such treatment.

“Arthritic condition” or “arthritic conditions” refers to a disease wherein inflammatory lesions are confined to the joints or any inflammatory conditions of the joints, most notably osteoarthritis and rheumatoid arthritis (Academic Press Dictionary of Science Technology; Academic Press; 1st edition, Jan. 15, 1992). The biphthalate salts of compound I of the present invention are also useful, alone or in combination, to treat or prevent arthritic conditions, such as Behcet's disease; bursitis and tendinitis; CPPD deposition disease; carpal tunnel syndrome; Ehlers-Danlos syndrome; fibromyalgia; gout; infectious arthritis; inflammatory bowel disease; juvenile arthritis; lupus erythematosus; lyme disease; marfan syndrome; myositis; osteoarthritis; osteogenesis imperfecta; osteonecrosis; polyarteritis; polymyalgia rheumatica; psoriatic arthritis; Raynaud's phenomenon; reflex sympathetic dystrophy syndrome; Reiter's syndrome; rheumatoid arthritis; scleroderma; and Sjogren's syndrome. An embodiment of the invention encompasses the treatment or prevention of an arthritic condition which comprises administering a therapeutically effective amount of the amorphous and crystalline forms, either alone or as a mixture, of the biphthalate salts of compound I. A subembodiment is the treatment or prevention of osteoarthritis, which comprises administering a therapeutically effective amount of a biphthalate salt of compound I. See: Cutolo M, Seriolo B, Villaggio B, Pizzorni C, Craviotto C, Sulli A. Ann. N.Y. Acad. Sci. June 2002;966:131-42; Cutolo, M. Rheum Dis Clin North Am November 2000;26(4):881-95; Bijlsma J W, Van den Brink H R. Am J Reprod Immunol October-December 1992;28(3-4):231-4; Jansson L, Holmdahl R.; Arthritis Rheum September 2001;44(9):2168-75; and Purdie D W. Br Med Bull 2000; 56(3):809-23. Also, see Merck Manual, 17th edition, pp. 449-451.

In another embodiment, the biphthalate salt of the compound of the present invention can be used to treat conditions in a female individual which are caused by androgen deficiency or which can be ameliorated by androgen replacement, including, but not limited to, osteoporosis, osteopenia, aging skin, glucocorticoid-induced osteoporosis, postmenopausal symptoms, periodontal disease, HIV-wasting, cancer cachexia, obesity, anemias, such as for example, aplastic anemia, muscular dystrophies, Alzheimer's disease, premature ovarian failure, cognitive decline, sexual dysfunction, depression, inflammatory arthritis and joint repair, atherosclerosis, and autoimmune disease, alone or in combination with other active agents. Treatment is effected by administration of a therapeutically effective amount of the biphthalate salt of compound I to a female individual in need of such treatment.

The biphthalate salt of compound I is also useful in the enhancement of muscle tone in mammals, such as for example, humans. The crystalline forms of the biphthalate salt of compound I can also be employed as adjuncts to traditional androgen depletion therapy in the treatment of prostate cancer to restore bone, minimize bone loss, and maintain bone mineral density. In this manner, they can be employed together with traditional androgen deprivation therapy, including GnRH agonists/antagonists, such as those disclosed in P. Limonta, et al., Exp. Opin. Invest. Drugs, 10: 709-720 (2001); H. J. Stricker, Urology, 58 (Suppl. 2A): 24-27 (2001); R. P. Millar, et al., British Medical Bulletin, 56: 761-772 (2000); and A. V. Schally et al., Advanced Drug Delivery Reviews, 28: 157-169 (1997). The solid state forms of the biphthalate salt of compound I can be used in combination with antiandrogens, such as flutamide, 2-hydroxyflutamide (the active metabolite of flutamide), nilutamide, and bicalutamide (Casodex™) in the treatment of prostate cancer.

Further, the present invention can also be employed in the treatment of pancreatic cancer, either for their androgen antagonist properties or as an adjunct to an antiandrogen, such as flutamide, 2-hydroxyflutamide (the active metabolite of flutamide), nilutamide, and bicalutamide (Casodex™).

The term “treating cancer” or “treatment of cancer” refers to administration to a mammal afflicted with a cancerous condition and refers to an effect that alleviates the cancerous condition by killing the cancerous cells, but also to an effect which results in the inhibition of growth and/or metastasis of the cancer.

The biphthalate salts of compound I can minimize the negative effects on lipid metabolism which may exhibit advantages over existing approaches for hormone replacement therapy in hypogonadic (androgen deficient) male individuals.

Additionally, the biphthalate salts of compound I can increase the number of blood cells, such as red blood cells and platelets, and can be used for treatment of hematopoietic disorders, such as aplastic anemia.

In one embodiment of the invention, therapeutically effective amounts of the amorphous, polymorphic, and pseudopolymorphic forms of the biphthalate salt of compound I, either alone or in combination, are administered to the mammal, to treat or improve disorders selected from enhancement of weakened muscle tone, osteoporosis, osteopenia, glucocorticoid-induced osteoporosis, periodontal disease, bone fracture, bone damage following bone reconstructive surgery, sarcopenia, frailty, aging skin, male hypogonadism, postmenopausal symptoms in women, atherosclerosis, hypercholesterolemia, hyperlipidemia, obesity, aplastic anemia and other hematopoietic disorders, pancreatic cancer, inflammatory arthritis and joint repair, HIV-wasting, prostate cancer, benign prostatic hyperplasia (BPH), cancer cachexia, Alzheimer's disease, muscular dystrophies, cognitive decline, sexual dysfunction, sleep apnea, depression, premature ovarian failure, and autoimmune disease.

In another embodiment, therapeutically effective amounts of the solid state forms of the biphthalate salt of compound I, either alone or in combination, can be used to treat or improve a disorder selected from weakened muscle tone, osteoporosis, osteopenia, glucocorticoid-induced osteoporosis, periodontal disease, bone fracture, bone damage following bone reconstructive surgery, sarcopenia, Alzheimer's disease, and frailty.

In another embodiment, the amorphous and crystalline forms of the compound in accordance with the invention can be used to treat or improve a disorder such as male hypogonadism, postmenopausal symptoms in women, atherosclerosis, hypercholesterolemia, hyperlipidemia, obesity, aplastic anemia and other hematopoietic disorders, pancreatic cancer, inflammatory arthritis and joint repair, HIV-wasting, prostate cancer, benign prostatic hyperplasia (BPH), cancer cachexia, muscular dystrophies, cognitive decline, sexual dysfunction, sleep apnea, depression, premature ovarian failure, and autoimmune disease.

The solid-state forms of the compound in accordance with the present invention can be administered in their enantiomerically pure form. Racemic mixtures can be separated into their individual enantiomers by any of a number of conventional methods. These include chiral chromatography, derivatization with a chiral auxiliary followed by separation by chromatography or crystallization, and fractional crystallization of diastereomeric salts.

As used herein, the biphthalate salts of compound I of the present invention, which functions as an “agonist” of the androgen receptor can bind to the androgen receptor and initiate a physiological or a pharmacological response characteristic of that receptor. The term “tissue-selective androgen receptor modulator” refers to an androgen receptor ligand that mimics the action of a natural ligand in some tissues but not in others. A “partial agonist” is an agonist which is unable to induce maximal activation of the receptor population, regardless of the amount of compound applied. A “full agonist” induces full activation of the androgen receptor population at a given concentration. The biphthalate salt of compound I which functions as an “antagonist” of the androgen receptor can bind to the androgen receptor and block or inhibit the androgen-associated responses normally induced by a natural androgen receptor ligand.

The term “therapeutically effective amount” means the amount the biphthalate salt of compound I that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts.

By “pharmaceutically acceptable” it is meant that the carrier, diluent or excipient must be compatible with the other ingredients of the formulation and not be deleterious to the recipient thereof.

The terms “administration of a compound” and “administering a compound” should be understood to mean providing a compound of the invention or a prodrug of a compound of the invention to the individual in need of treatment.

By the term “modulating a function mediated by the androgen receptor in a tissue selective manner” it is meant modulating a function mediated by the androgen receptor selectively (or discriminately) in anabolic (bone and/or muscular) tissue (bone and muscular) in the absence of such modulation at androgenic (reproductive) tissue, such as the prostate, testis, seminal vesicles, ovary, uterus, and other sex accessory tissues. In one embodiment, the function of the androgen receptor in anabolic tissue is activated whereas the function of the androgen receptor in androgenic tissue is blocked or suppressed. In another embodiment, the function of the androgen receptor in anabolic tissue is blocked or suppressed whereas the function of the androgen receptor in androgenic tissue is activated.

The administration of an amorphous or crystalline form of the biphthalate salt of compound I, in order to practice the present methods of therapy, is carried out by administering an effective amount of the compound to the patient in need of such treatment or prophylaxis. The need for a prophylactic administration according to the methods of the present invention is determined via the use of well-known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient can concomitantly require, and other factors in the physician's judgment.

If formulated as a fixed dose, such combination products employ the amorphous and crystalline forms of the biphthalate salt of compound I within the dosage range described below and the other pharmaceutically active agent(s) within its approved dosage range. The amorphous and crystalline forms of the compound can alternatively be used sequentially with known pharmaceutically acceptable agent(s) when a combination formulation is inappropriate.

Generally, the daily dosage of an amorphous or crystalline form of the biphthalate salt of compound I can be varied over a wide range from about 0.01 to about 1000 mg per adult human per day. For example, dosages range from about 0.1 to about 200 mg/day. For oral administration, the compositions can be provided in the form of tablets containing from about 0.01 to about 1000 mg, such as for example, 0.01, 0.05, 0.1, 0.5, 1.0, 2.5, 3.0, 5.0, 6.0, 10.0, 15.0, 25.0, 50.0, 75, 100, 125, 150, 175, 180, 200, 225, and 500 milligrams of the active ingredient for the symptomatic adjustment of the dosage to the mammal to be treated.

The dose can be administered in a single daily dose or the total daily dosage can be administered in divided doses of two, three or four times daily. Furthermore, based on the properties of the individual compound selected for administration, the dose can be administered less frequently, e.g., weekly, twice weekly, monthly, etc. The unit dosage will, of course, be correspondingly larger for the less frequent administration.

When administered via intranasal routes, transdermal routes, by rectal or vaginal suppositories, or through an intravenous solution, the dosage administration will, of course, be continuous rather than intermittent throughout the dosage regimen.

Exemplifying the invention is a pharmaceutical composition comprising any of the amorphous and crystalline forms of the compound, either alone or in combination, described above and a pharmaceutically acceptable carrier. The carrier must be pharmaceutically acceptable in the sense of being compatible with the other ingredients of the formulation and not being deleterious to the recipient subject of the formulation.

The present invention, therefore, further provides a pharmaceutical formulation comprising amorphous or crystalline forms of the biphthalate salt of compound I together with a pharmaceutically acceptable carrier thereof. The formulations include those suitable for oral, rectal, intravaginal, intranasal, topical and parenteral (including subcutaneous, intramuscular and intravenous administration). In one embodiment, the formulations are those suitable for oral administration.

Suitable topical formulations of the biphthalate salt of compound I include transdermal devices, aerosols, creams, solutions, ointments, gels, lotions, dusting powders, and the like. The topical pharmaceutical compositions containing the compounds of the present invention ordinarily include about 0.005% to about 5% by weight of the active compound in admixture with a pharmaceutically acceptable vehicle. Transdermal skin patches useful for administering the compounds of the present invention include those well known to those of ordinary skill in that art.

The formulations can be presented in a unit dosage form and can be prepared by any of the methods known in the art of pharmacy. All methods include the step of bringing the active compound in association with a carrier, which constitutes one or more ingredients. In general, the formulations are prepared by uniformly and intimately bringing the active compound in association with a liquid carrier, a waxy solid carrier, or a finely divided solid carrier, and then, if needed, shaping the product into the desired dosage form.

Formulations of the present invention suitable for oral administration can be presented as discrete units such as capsules, cachets, tablets or lozenges, each containing a predetermined amount of the active compound; as a powder or granules; or a suspension or solution in an aqueous liquid or non-aqueous liquid, e.g., a syrup, an elixir, or an emulsion.

A tablet can be made by compression or molding, optionally with one or more accessory ingredients. Compressed tablets can be prepared by compressing in a suitable machine the active compound in a free flowing form, e.g., a powder or granules, optionally mixed with accessory ingredients, e.g., binders, lubricants, inert diluents, disintegrating agents or coloring agents. Molded tablets can be made by molding in a suitable machine a mixture of the active compound, preferably in powdered form, with a suitable carrier. Suitable binders include, without limitation, starch, gelatin, natural sugars such as glucose or beta-lactose, corn sweeteners, natural and synthetic gums such as acacia, tragacanth or sodium alginate, carboxymethyl-cellulose, polyethylene glycol, waxes and the like. Non-limiting representative lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

Oral liquid forms, such as syrups or suspensions in suitably flavored suspending or dispersing agents such as the synthetic and natural gums, for example, tragacanth, acacia, methyl cellulose and the like, can be made by adding the active compound to the solution or suspension. Additional dispersing agents which can be employed include glycerin and the like.

Formulations for vaginal or rectal administration can be presented as a suppository with a conventional carrier, i.e., a base that is nontoxic and nonirritating to mucous membranes, compatible with the biphthalate salt of compound I, and is stable in storage and does not bind or interfere with the release of the amorphous or crystalline forms of the biphthalate salt of compound I. Suitable bases include: cocoa butter (theobroma oil), polyethylene glycols (such as carbowax and polyglycols), glycol-surfactant combinations, polyoxyl 40 stearate, polyoxyethylene sorbitan fatty acid esters (such as Tween, Myrj, and Arlacel), glycerinated gelatin, and hydrogenated vegetable oils. When glycerinated gelatin suppositories are used, a preservative such as methylparaben or propylparaben can be employed.

Topical preparations containing the active drug component can be admixed with a variety of carrier materials well known in the art, such as, e.g., alcohols, aloe vera gel, allantoin, glycerine, vitamin A and E oils, mineral oil, PPG2 myristyl propionate, and the like, to form, e.g., alcoholic solutions, topical cleansers, cleansing creams, skin gels, skin lotions, and shampoos in cream or gel formulations.

The amorphous and crystalline forms of the 2:1 compound I:biphthalate salt of N-(3H-imidazo[4,5-B]pyridin-2-ylmethyl)-2-fluoro-4-methyl-3-oxo-4-aza-5-alpha-androst-1-en-17-beta-carboxamide of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholines.

The SARM of the present invention can also be delivered by the use of monoclonal antibodies as individual carriers to which the compound molecules are coupled. The compound of the present invention can also be coupled with soluble polymers as targetable drug carriers. Such polymers can include polyvinyl-pyrrolidone, pyran copolymer, polyhydroxypropylmethacrylamide-phenol, polyhydroxy-ethylaspartamidephenol, or polyethylene-oxide polylysine substituted with palmitoyl residues. Furthermore, the compound of the present invention can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Formulations suitable for parenteral administration include formulations that comprise a sterile aqueous preparation of the active compound which can be isotonic with the blood of the recipient. Such formulations suitably comprise a solution or suspension of a compound that is isotonic with the blood of the recipient subject. Such formulations can contain distilled water, 5% dextrose in distilled water, or saline and the active compound. Useful formulations also comprise concentrated solutions or solids comprising the solid state form of the biphthalate salt of compound I, which on dilution with an appropriate solvent give a solution suitable for parenteral administration.

Abbreviations used in the Description of the Preparation of the Compounds of the Present Invention:
AcOH Acetic acid
CBZ-glycine Protected from of glycine (C₁₀H₁₁NO₄)
DHT Dihydrotestosterone
DMEM Dulbecceo modified eagle media
DMSO Dimethyl sulfoxide
DMF N,N-Dimethylformamide
EDC 1-(3-Dimethylaminopropyl)3-ethylcarbodiimide HCl
EDTA Ethylenediaminetetraacetic acid
EtOAc Ethyl acetate
EtOH Ethanol
FBS Fetal bovine serum
FCS Fetal calf serum
HAP Hydroxyapatite
HEPES (2-Hydroxyethyl)-1-piperazineethanesulfonic acid
HOAt 1-hydroxy-7-azabenzotriazole
HOBt N-hydroxybenzotriazole
HPLC High-performance liquid chromatography
LCMS Liquid chromatography/mass spectroscopy
LCWP Weight percent by liquid chromatography
LDA Lithium diisopropylamide
MeOH Methanol
NMM N-methylmorpholine
n-Bu4NI Tetra-n-butylammonium iodide
Pd/C Palladium/carbon
Rt or rt Room temperature
TEGM Binding buffer
THF Tetrahydrofuran

The compounds of this invention may be prepared by employing reactions as shown in the following schemes, in addition to other standard manipulations that are known in the literature or exemplified in the experimental procedures.

### EXAMPLE 1

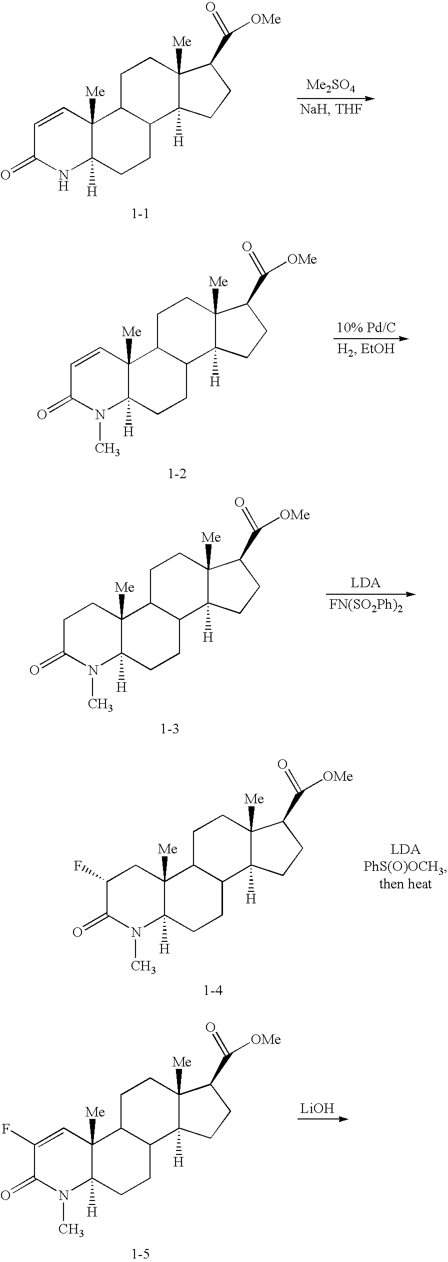

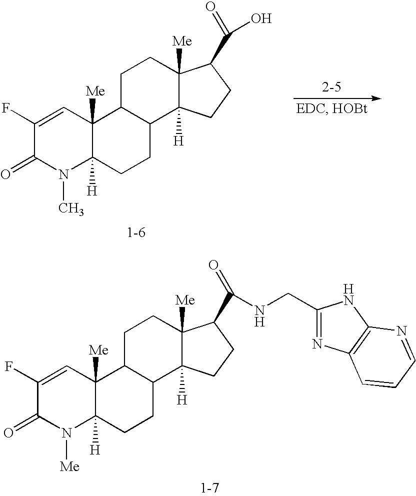

### I. Formation of 2-(Ammoniomethyl)-3H-imidazo[4,5-b]pyridin-4-ium dichloride (2-5)

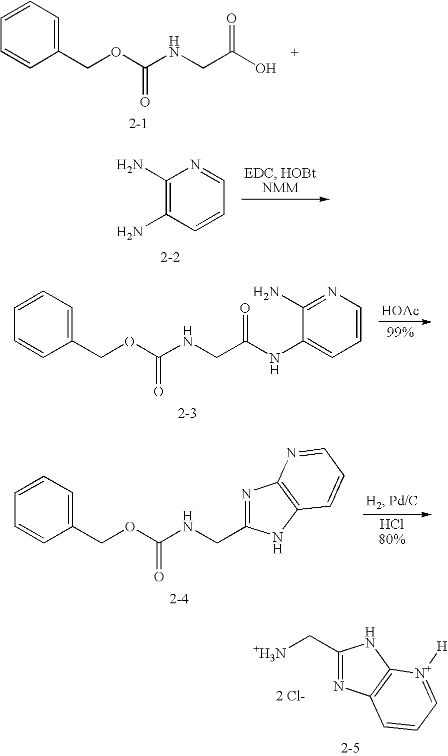

### Step I.A: N-(2-aminopyridin-3-yl)-N′-carboxybenzylglycinamide (2-3)

A mixture of 2,3-diaminopyridine, (2-1, 20.866 g, 191.2 mmol), Cbz-glycine (2-2, 40 g, 191.2 mmol), EDC (43.93 g, 229.44 mmol), HOAT (26.02 g, 191.2 mmol) and NMM (82.12 mL, 764.81 mmol) in DMF (300 mL) was stirred for 20 hr. The mixture was diluted with H₂O (500 mL) and extracted with EtOAc (3×500 mL). The combined organic portions were washed with sat. NaHCO₃, brine, and dried over MgSO₄ and then concentrated to give the product 2-3 as a brown solid. ¹H NMR (500 MHz, CD₃OD) 7.82 (d, 1H, J=5 Hz), 7.49 (d, 1H, J=8 Hz), 7.31 (m, 5H), 6.65 (m, 1H), 5.12 (s, 2H). HRMS (ES, M+1) calc'd 301.1295, found 301.1296.

### Step I.B: Benzyl 3H-imidazo[4,5-b]pyridin-2-ylmethylcarbamate (2-4)

The aminopyridine 2-3 (46 g, 153 mmol) was dissolved in 300 mL of AcOH and heated to 120° C. for 20 hours. The reaction mixture was cooled to room temperature and concentrated to give the desired product 2-4 as the acetate salt. ¹H NMR (500 MHz, CD₃OD) 8.32 (m, 1H), 7.95 (m, 1H), 7.34 (m, 6H), 7.08 (m, 1H), 5.14 (s, 2H), 4.87 (s, 2H). HRMS (ES, M+1) calc'd 283.1190, found 283.1192.

### Step I.C: 2-(Ammoniomethyl)-3H-imidazo[4,5-b]pyridin-4-ium dichloride (2-5)

To a mixture of 2-4 (52 g, 151.88 mmol) in 1000 mL of 1:1 AcOH/MeOH was added 20 g 10% Pd/C. The reaction mixture was stirred under 1 atm H₂ for 20 hours. The mixture was filtered through a celite pad, concentrated, and then azeotroped with dioxane to give tan oil. The semisolid or thick oil was suspended in 200 mL dioxane and 200 mL 4.0 M HCl/dioxane was added to produce a tan suspension. The solid was collected, washed with dioxane (200 mL) and dried in vacuo to give 2-5 as a tan solid. ¹H NMR (500 MHz, CD₃OD) 8.75 (d, 1H, J=8 Hz), 8367 (d, 1H, J=6 Hz), 7.85 (m, 1H), 4.88 (s, 2H). HRMS (ES, M+1) calc'd 149.0822, found 149.0812.

### II. Amorphous Form of N-(3H-imidazo[4,5-B]pyridin-2-ylmethyl)-2-fluoro-4-methyl-3-oxo-4-aza-5-alpha-androst-1-en-17-beta-carboxamide (1-7)

### Step II.A: 4-methyl-3-oxo-4-aza-5α-androst-1-ene-17β-carboxylic acid methyl ester (1-2)

A mixture of 1-1 (J. Med. Chem. 29 2298-2315 (1986)) (120 g, 362.04 mmol), 60% NaH (18.7 g, mmol), dimethylsulfate (68.50 g, 543.05 mmol), and THF (1200 mL) was stirred at ambient temperature for 14 hours. Warmed to 60° C. for 2.0 hours and then the reaction was quenched by the slow addition of 40 mL conc. HCl while purging the headspace with nitrogen. Evaporated to ⅕ volume and then diluted with 1500 mL water. The solid was collected, washed with water, hexanes and dried in vacuo to give 1-2 as a pale yellow solid.

### Step II.B: 4-methyl-3-oxo-4-aza-5α-androstane-17β-carboxylic acid methyl ester (1-3)

To a solution of 1-2 (20 g, 57.9 mmol) in EtOH (100 mL) was added 10% Pd/C (5.0 g) and the mixture was stirred under 1 atm H₂ for 14 hours. The mixture was filtered through a celite pad, concentrated and then dried in vacuo to give 1-3 as a white solid.

### Step II.C: 2α-Fluoro-4-methyl-3-oxo-4-aza-5α-androstane-17β-carboxylic acid methyl ester (1-4)

To a solution of 1-3 (12 g, 34.53 mmol) in THF (100 mL) at −78° C. was added a solution of 1.5 M LDA in THF (27.6 mL, 41.44 mmol) dropwise over 20 min and then stirred 1 h. A solution of FN(SO₂Ph)₂ (13.07 g, 41.44 mmol) in THF (40 mL) was then added over 20 min. After 30 min, the cooling bath was removed and the reaction was stirred for 14 h. Et₂O was added, and the mixture was washed with water, saturated aqueous sodium hydrogencarbonate, brine, dried (MgSO₄) and then concentrated. Chromatography on silica gel (hexanes to EtOAc as eluent) gave 1-4 as a colorless solid.

MS calculated M+H: 366, found 366.1.

### Step II.D: 2-Fluoro-4-methyl-3-oxo-4-aza-5α-androst-1-ene-17β-carboxylic acid methyl ester (1-5)

To a solution of 1-4 (30 g, 82.1 mmol) in THF (400 mL) at −78° C. was added a solution of 1.5 M LDA in THF (71.1 mL, 107 mmol) dropwise over 30 min and then stirred 1 h. Methyl benzenesulfinate (19.23 g, 123 mmol) was then added over 15 min. After 30 min, the cooling bath was removed and the reaction was stirred for 1 h. Et₂O was added, and the mixture was washed with water, saturated aqueous sodium hydrogencarbonate, brine, dried (MgSO₄) and then concentrated. The residue was dissolved in toluene (200 mL) and heated at reflux for 2 h. Solvent evaporation and chromatography of the residue on silica gel (hexanes to 50% EtOAc/hexanes as eluent) gave 1-5 as a pale yellow solid.

MS calculated M+H: 364, found 364.1.

### Step II.E: 2-Fluoro-4-methyl-3-oxo-4-aza-5α-androst-1-ene-17β-carboxylic acid (1-6)

To a solution of 1-5 (6.2 g, 17.1 mmol) in 1,4-dioxane (50 mL) was added a solution of lithium hydroxide (1.07 g, 25.6 mmol) in water (30 mL), and the mixture heated at 100° C. for 3 h. After cooling, the mixture was diluted with ethyl acetate, separated, and the organics washed with 1N HCl, brine, dried (MgSO₄) and then concentrated to give 1-6 as a pale yellow solid.

MS calculated M+H: 350, found 350.

### Step II.F: N-(3H-imidazo[4,5-b]pyridin-2-ylmethyl)-2-fluoro-4-methyl-3-oxo-4-aza-5α-androst-1-en-17β-carboxamide (1-7)

A mixture of 1-6 (15 g, 42.9 mmol), EDC (9.88 g, 51.5 mmol), HOBt (6.96 g, 51.510 mmol), NMM (18.9 mL, 171.70 mmol) and 2-5 (10.43 g, 47.217 mmol) in DMF (200 mL) was heated to 40° C. for 4.0 hours and then stirred at ambient temperature for 14 h. The mixture was diluted with water, filtered, and the solids washed with water and then dried under vacuum. Chromatography on silica gel (hexanes to 70:25:5 CHCl₃/EtOAc/MeOH as eluent) gave 1-7 as a colorless amorphous solid.

¹H NMR (500 MHz, CDCl₃) 8.45 (m, 1H), 7.95 (m, 1H), 7.23 (m, 1H), 6.14 (d, 1H, J=8 Hz), 4.72 (m, 2H), 3.40 (dd, 1H, J=4 Hz, 13 Hz), 2.94 (s, 3H), 2.23 (m, 2H), 1.96 (m, 2H), 1.53-1.86 (m, 7H), 1.25-1.39 (m, 3H), 1.12 (m, 1H), 1.04 (m, 2H), 0.95 (s, 3H), 0.68 (s, 3H). MS calculated M+H: 480.2770 found 480.2740.

### EXAMPLE 2

A large quantity of Form A (wet cake of N-(3H-imidazo[4,5-B]pyridin-2-ylmethyl)-2-fluoro-4-methyl-3-oxo-4-aza-5-alpha-androst-1-en-17-beta-carboxamide) was generated by dissolving the crystalline free base (Form E) in pH 3—potassium biphthalate and sulfuric acid mixture—for 10 days at RT. The slurry was filtered and washed with copious amount of D.I. water to wash off residual ions. The wet cake (Form A) was analyzed by x-ray and was then vacuum dried at 37° C. for 3 days. Upon drying, a change in x-ray pattern was observed. The dry cake (Form B) was then analyzed by differential scanning calorimetry (DSC) and thermogravimetry (TG). The TG analysis was performed under a nitrogen flow at a heating rate of 10° C./min, and the DSC was performed under nitrogen flow at a heating rate of 10° C./min in a covered pan. The hygroscopicity of Form B was determined using a symmetric vapor sorption analyzer. The experiment includes an initial drying step at 60° C., followed by measurements of water sorption at 25° C. for percent RH values from 5% to 95% in 10% intervals. The data are reported as percent weight gain as a function of percent RH. Form B was also analyzed for weight percent by liquid chromatography (LCWP) by high performance liquid chromatography (HPLC).

### Assays

### In Vitro and In Vivo Assays for SARM Activity Identification of Compounds

The compounds exemplified in the present application exhibit activity in one or more of the assays reported in U.S. Pat. No. 7,186,838, and are incorporated by reference in their entirety.

What is claimed is:

1. A crystalline 2:1 compound I:biphthalate salt of N-(3H-imidazo[4,5-B]pyridin-2-ylmethyl)-2-fluoro-4-methyl-3-oxo-4-aza-5-alpha-androst-1-en-17-beta-carboxamide.

2. The crystalline Form B of claim 1.

3. The crystalline dihydrate of the 2:1 compound I: biphthalate salt of N-(3H-imidazo[4,5-B]pyridin-2-ylmethyl)-2-fluoro-4-methyl-3-oxo-4-aza-5-alpha-androst-1-en-17-beta-carboxamide of claim 1.

4. The dihydrate crystalline Form B of the 2:1 compound I: biphthalate salt of N-(3H-imidazo[4,5-B]pyridin-2-ylmethyl)-2-fluoro-4-methyl-3-oxo-4-aza-5-alpha-androst-1-en-17-beta-carboxamide of claim 3, having an X-ray powder diffraction pattern with characteristic d-spacings of 5.65±0.1, 4.62±0.1, and 4.40±0.1 angstroms.

5. The dihydrate crystalline Form B of the 2:1 compound I: biphthalate salt of N-(3H-imidazo[4,5-B]pyridin-2-ylmethyl)-2-fluoro-4-methyl-3-oxo-4-aza-5-alpha-androst-1en-17-beta-carboxamide of claim 4, which is further characterized as having X-ray powder diffraction d-spacings of 7.41±0.1, 4.10±0.1, and 3.38±0.1 angstroms.

6. The dihydrate crystalline Form B of the 2:1 compound I: biphthalate salt of N-(3H-imidazo[4,5-B]pyridin-2-ylmethyl)-2-fluoro-4-methyl-3-oxo-4-aza-5-alpha-androst-1-en-17-beta-carboxamide of claim 5, which is still further characterized as having X-ray powder diffraction d-spacings of 3.84±0.1, 3.72±0.1 and 3.24±0.1 angstroms.

7. The dihydrate crystalline Form B of claim 6, which is further characterized as having a melting point about 168.7±0.5° C.

8. The dihydrate crystalline Form B of claim 7, which is further characterized as having a solid-state Carbon-13 nuclear magnetic resonance (NMR) spectrum with signals having chemical shift values of 13.8±0.1, 44.4±0.1, and 119.4±0.1 p.p.m.

9. The dihydrate crystalline Form B of claim 8, which is further characterized as having signals with chemical shift values of 28.7±0.1, 56.2±0.1, and 173.3±0.1 p.p.m.

10. The dihydrate crystalline Form B of claim 9, which is even further characterized as having signals with chemical shift values of 38.6±0.1, 22.1±0.1, and 158.9±0.1 p.p.m

11. A method of making a crystalline dihydrate 2:1 biphthalate salt Form B, of N-(3H-imidazo[4,5-B]pyridin-2-ylmethyl)-2-fluoro-4-methyl-3-oxo-4-aza-5-alpha-androst-1-en-17-beta-carboxamide comprising:
a) dissolving N-(3H-imidazo[4,5-B]pyridin-2-ylmethyl)-2-fluoro-4-methyl-3-oxo-4-aza-5-alpha-androst-1-en-17-beta-carboxamide in a biphthalate solution having a pH ranging from about 2 to about 9;
b) isolating the solids;
c) rinsing the solids with a solvent; and
d) drying the solids.

12. The method according to claim 11, wherein the biphthalate solution is selected from ammonium biphthalate and potassium biphthalate.

13. The method according to claim 12, wherein the biphthalate solution includes an acid selected from sulfuric acid and formic acid.

14. The method according to claim 13, wherein at least one said solvent is water.

15. The method according to claim 14, wherein isolating the solids further comprises filtering of the solids via a vacuum filter and washing the solids with at least one wash with a solvent consisting of an aqueous solution.

16. The method according to claim 15 wherein the aqueous solution is water.

17. A pharmaceutical composition comprising a therapeutically effective amount of at least one substantially pure crystalline form of the 2:1 compound I:biphthalate salt of N-(3H-imidazo[4,5-B]pyridin-2-ylmethyl)-2-fluoro-4-methyl-3-oxo-4-aza-5-alpha-androst-1-en-17-beta-carboxamide and at least one pharmaceutically acceptable carrier.

18. The pharmaceutical composition of claim 17, wherein said substantially pure crystalline form is selected from dihydrate Form B, anhydrous Form C, tetrahydrate Form D, and mixtures thereof.
